(19) United States
(12) Patent Application Publication
(10) Pub. No.: US 2012/0150242 A1
(43) Pub. Date: Jun. 14, 2012

(54) METHOD FOR PLACING SPINAL IMPLANTS

(76) Inventor: Richard Mannion, ArlingtonHeights, IL (US)

(21) Appl. No.: 12/967,237

(22) Filed: Dec. 14, 2010

(51) Int. Cl. A61B 17/56

(52) U.S. Cl. 606/86 A

(57) ABSTRACT

A process for utilizing a spinal implant guide made using a diagnostic imaging tool to facilitate implantation of a device in the spine. The spinal implant guide preferably has a body designed to conform to certain predetermined, patient-specific anatomical vertebral landmarks. The guide may be fitted to the anatomical vertebral landmarks pre-operatively and/or during a surgical procedure using 3-point fixation technique to thereby substantially demarcate an optimal alignment, trajectory and angulation for the spinal implant device.

### BACKGROUND OF THE INVENTION

The present inventions generally relate to methods for augmenting the structural stability of the spine. More specifically, methods are disclosed and claimed for placing pedicle screws in the spine using synthetic spine models creating using diagnostic imaging tools, and surgical placement techniques that can be replicated.

In human anatomy, the vertebral column (backbone or spine) is a column usually consisting of 24 articulating vertebrae, and multiple fused vertebrae in the sacrum and the coccyx. The 24 articulating vertebrae are grouped under the names cervical (7 vertebrae in the neck), thoracic (12 vertebrae in the chest area) and lumbar (5 vertebrae in the lower back), according to the regions they occupy. The spine is situated in the dorsal aspect of the torso, separated by intervertebral discs. It houses and protects the spinal cord in its spinal canal.

Spinal fusion procedures involving the implantation of pedicle screws have steadily increased over the past decade because of demonstrated improvement in biomechanical stability of the spine. Since pedicle screws traverse all three columns of the vertebrae (anterior, middle and posterior), they can rigidly stabilize both the ventral and dorsal aspects of the spine. The pedicle also represents the strongest point of attachment of the spine, permitting significant forces to be applied to the spine without failure of the bone-metal junction.

However, current methods of spinal fusion carry a risk of vascular, visceral and neurological injury caused by inaccurate placement of pedicle screws, or inappropriately-sized instrumentation, which may lead to patient paralysis or even fatality. For example, given infirmities inherent in known techniques, the literature suggests pedicle screw misplacement may be as high as 40%.

3D spine templating software, such as the software developed by the Biomedical Imaging Resource at Mayo Clinic, allows the surgeon to virtually place pedicle screws using pre-operative 3D CT image data. Using this software, a patient-specific 3D anatomic model may be produced using a commercial rapid prototyping system. The pre-surgical plan and the patient-specific model may then be used in the procedure room to provide real-time visualization and quantitative guidance for accurate placement of each pedicle screw. However, this and similar methods still place a premium on surgeon experience and skill to introduce a guide wire and screw into the spine in the proper location and with the proper orientation and trajectory.

A primary cause of misplacement is the surgeon's inability to accurately align the pedicle screw and provide it with an appropriate trajectory given the particular spinal geometry involved. The goal is to achieve 5-wall bony continuity, i.e., the pedicle screw is completely surrounded on all sides by bone, and the bottom of the crew abuts and is located within a bony floor. From an anatomical perspective, providing 5-wall bony continuity with proper trajectory will optimally place the fixation device within the pedicle, regardless of the design characteristics of the implant. Misaligned pedicle screw problems range from minor issues to more serious problems such as a lack of spinal integrity and resulting paralysis or other serious health issues.

Typical placement of spinal implant instrumentation involves passing the implant (e.g., a feeler gauge, bur, pedicle screw, etc.) through the facet and also through the pedicle; the tip of the implant (e.g., the pedicle screw) may then be fastened to the vertebral body using free-hand technique, thus securing the facet to the vertebral body (see FIGS. 7-8). This has been typically done by spinal surgeons using free-hand trajectory analysis, without guides or templates.

What is needed is a surgical technique that can be replicated and that takes proper advantage of a patient-specific, anatomically-correct surgical guide made using modern diagnostic imaging tools, in order to safely, accurately and consistently place spinal implants with an optimal trajectory.

### SUMMARY OF THE INVENTION

The objects mentioned above, as well as other objects which will be recognized by those of ordinary skill in the art, are solved by the present invention, which overcomes disadvantages of prior spinal instrumentation placement methods, while providing new advantages not believed associated with such prior methods.

According to the present invention, 3D diagnostic imaging technology, such as computerized tomography (CT), X-Ray or MRI scans, may be used to make a patient-specific, anatomically correct guide which, when properly placed as described here, will provide the surgeon with a predetermined trajectory for the pedicle implant. The guide may then be manufactured using an appropriate rapid prototyping process, sterilized, and then used pre-operatively in the planning of the surgical procedure (during which the guide may be manipulated relative to a patient's bone model) and/or intra-operatively during the surgical procedure by placing it over the patient's anatomy in-situ to drill a pilot hole and/or manually insert a guide wire in the optimal trajectory. The guide may be custom-disposable or adjustable and reusable, with that choice depending on its specifically designed purpose, and its cost and material make-up. The guide may serve as a drill guide or guide wire guide for targeting the pedicle in the optimal trajectory. The device may be made from a pre-op diagnostic image scan of the patient, for example, and delivered for use in the planning and/or the completion of the surgery.

Proper placement of the guide forms an important part of the present invention, which incorporates the principles of 3-point fixation. 3-point fixation theory has been commonly used to provide a fulcrum for control and correction of a deformity in the treatment of fractures. Despite this fact, and despite an absence of literature recognition, present inventor has discovered, that contrary to previous theory, the 3-point fixation technique may be advantageously used not to control and/or correct a spinal deformity, but rather to accept the deformity and to use the 3-point fixation technique only for guidance purposes. This enables the surgeon to properly control both the location and trajectory of the spinal implant—and to do so despite any pre-existing deformities, using pre-determined, patient-specific, 3-point fixation to determine optimal implant trajectory in 3 planes. Thus, using a custom-made, single-use or multiple-use spinal guide, placed utilizing 3-point fixation techniques (such as by using the medial and lateral boundaries of a spinal facet, and an adjacent transverse process of the spine), the guide may be used to place a spinal implant with an optimal trajectory in three planes. This novel method of using 3-point fixation technique to determine and to substantially demarcate the alignment, trajectory and angulation of implanted spinal instrumentation has been used by the present inventor to consistently and accurately place spinal instrumentation, in a fashion which may be replicated by even less skilled practitioners, in a consistent and reproducible manner.

In a preferred embodiment, a process is provided for using a spinal implant guide to facilitate the proper location and positioning of a device to be implanted in the spine. The guide may be reusable or disposable, depending on user preference, the economics of the material chosen to make the guide, etc. The guide is preferably sized and shaped to conform or fit an individual patient's vertebral anatomy, and may be made using biofeedback information provided by an appropriate diagnostic imaging tool, such as a CT, X-Ray or MRI machine, which preferably provides a three-dimensional reconstruction of anatomical images of selected portions of the spine. The guide preferably has a body sized and shaped to conform to certain predetermined, patient-specific anatomical vertebral landmarks. The guide may be fit to the anatomical vertebral landmarks during a surgical procedure using 3-point fixation technique so that the guide substantially demarcates an optimal alignment, trajectory and angulation for the spinal implant device. The guide, or an earlier version of the guide, may be used pre-operatively to fit to a patient's vertebral model, to analyze the proper fit of the guide, and to determine whether it needs to be modified for intra-operative use. In the lumbar region of the spine, the following anatomical landmarks may be used to accomplish 3-point fixation of the guide: medial and lateral boundaries of a spinal facet joint, and an adjacent transverse process of the spine. In other regions of the spine, artisans will understand that other anatomical landmarks may be used to accomplish 3-point fixation of the guide, and that the choice of all 3 points of fixation is variable dependent on the area of the spine to be instrumented.

Spinal implant devices which may be implanted in an optimal location using the guide of the present invention include but are not limited to one or more of the following: a guide wire; a bur; a feeler gauge; or a pedicle screw.

In one preferred process embodiment, the spinal implant guide may be fitted to the relevant human vertebral anatomy using 3-point fixation to optimally locate a guide wire inserted through the facet and into the pedicle. The guide wire may then be withdrawn and a pedicle screw may be inserted within the passageway vacated by the guide wire.

In one preferred embodiment, the spinal implant guide may include a sleeve guide and a sleeve shaped to at least partially fit within the sleeve guide, to facilitate entry and placement of the spinal implant device. Wherein among different sleeves which may be provided and used for different patients, an outer diameter of the sleeve may preferably be constant for such different sleeves, whereas an inner diameter of the sleeve may be allowed to vary to fit individual patient anatomies.

### DEFINITION OF CLAIM TERMS

The following terms are used in the claims of the patent as filed and are intended to have their broadest meaning consistent with the requirements of law. Where alternative meanings are possible, the broadest meaning is intended. All words used in the claims are intended to be used in the normal, customary usage of grammar and the English language.

“Spinal implant” means any device which surgeons may choose to implant in the spine, such as but not limited to feeler gauges, guide wires, burs, pedicle screws, etc.

“Diagnostic imaging tool” means devices which may be used to provide images of the vertebral anatomy, such as but not limited to CT, X-Ray and MRI machines and devices.

“An optimal” as used in the claim phrase “to thereby substantially demarcate an optimal alignment, trajectory and angulation for the spinal implant device” means one of the several such spinal implant device locations which a surgeon may deem “optimal” to provide 3-point fixation of the device relative to the patient's relevant anatomical, vertebral landmarks, while avoiding unintended impingement of the device on adjacent spinal, vascular or neurological elements.

### BRIEF DESCRIPTION OF THE DRAWINGS

The novel features which are characteristic of the invention are set forth in the appended claims. The invention itself, however, together with further objects and attendant advantages thereof, can be better understood by reference to the following description taken in connection with the accompanying drawings, in which:

FIG. 1 is a perspective view of a partial spinal model, with the arrows referring to medial and lateral borders of the spinal facet joint;

FIG. 2 is a top and side perspective view of one preferred embodiment of the guide of the present invention, showing an (e.g., guide wire) implant placed into the spinal vertebrae using a guide of the present invention;

FIG. 3 is a top, perspective view of FIG. 2;

FIG. 4 is a side, perspective view of FIG. 2;

FIG. 5 is a perspective view showing a dual guide facilitating placement of two pedicle screws into adjacent vertebrae of the human spine;

FIG. 6 is a posterior, perspective view of the human lumbar and sacral spine and pelvis showing a custom guide of the present invention facilitating the placement of multiple pedicle screws;

FIG. 7 is a side, perspective view of a human spine showing several pedicle implants placed in the lower lumbar region using prior free-hand technique; and

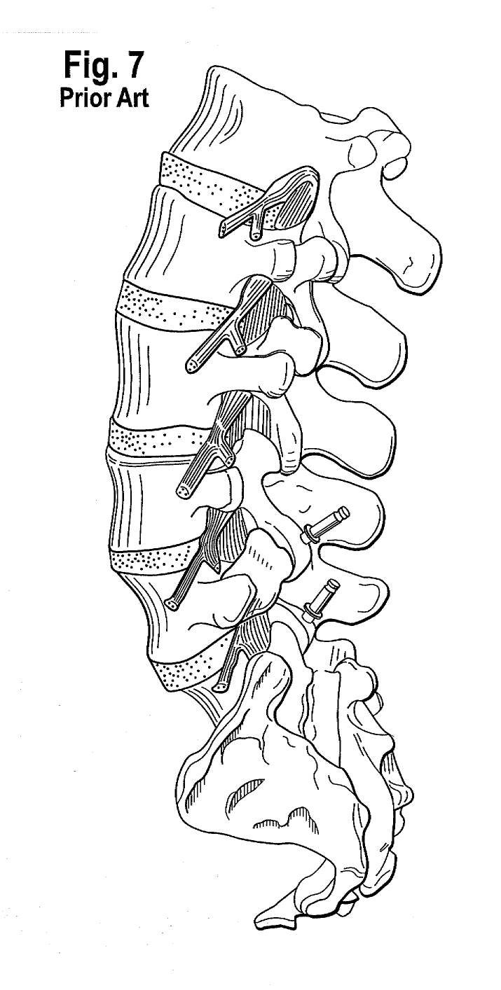

FIG. 8 is a posterior, perspective view of FIG. 7.

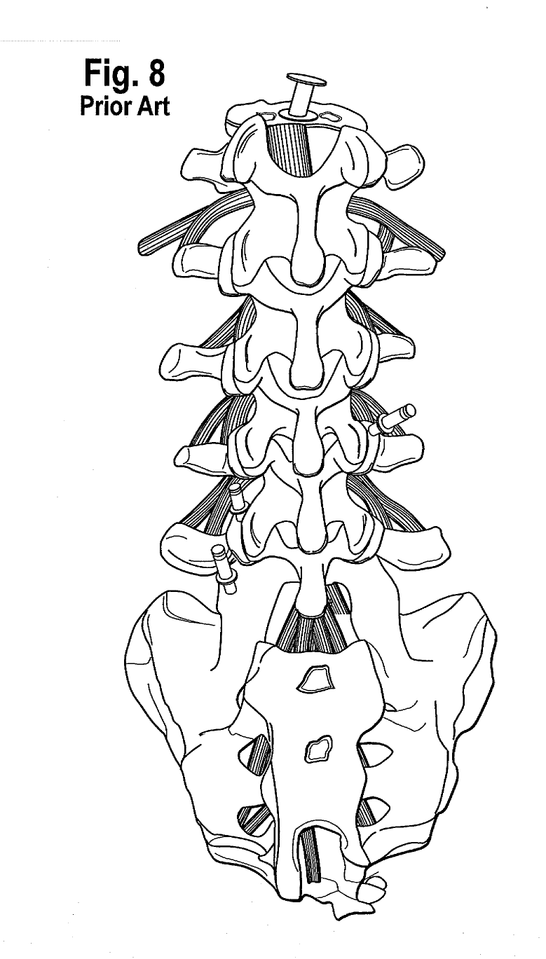

The components in the drawings are not necessarily to scale, emphasis instead being placed upon clearly illustrating the principles of the present invention. In the drawings, like reference numerals designate corresponding parts throughout the several views.

### DETAILED DESCRIPTION OF THE PREFERRED EMBODIMENTS

Set forth below is a description of what are believed to be the preferred embodiments and/or best examples of the invention claimed. Future and present alternatives and modifications to this preferred embodiment are contemplated. Any alternatives or modifications which make insubstantial changes in function, in purpose, in structure, or in result are intended to be covered by the claims of this patent.

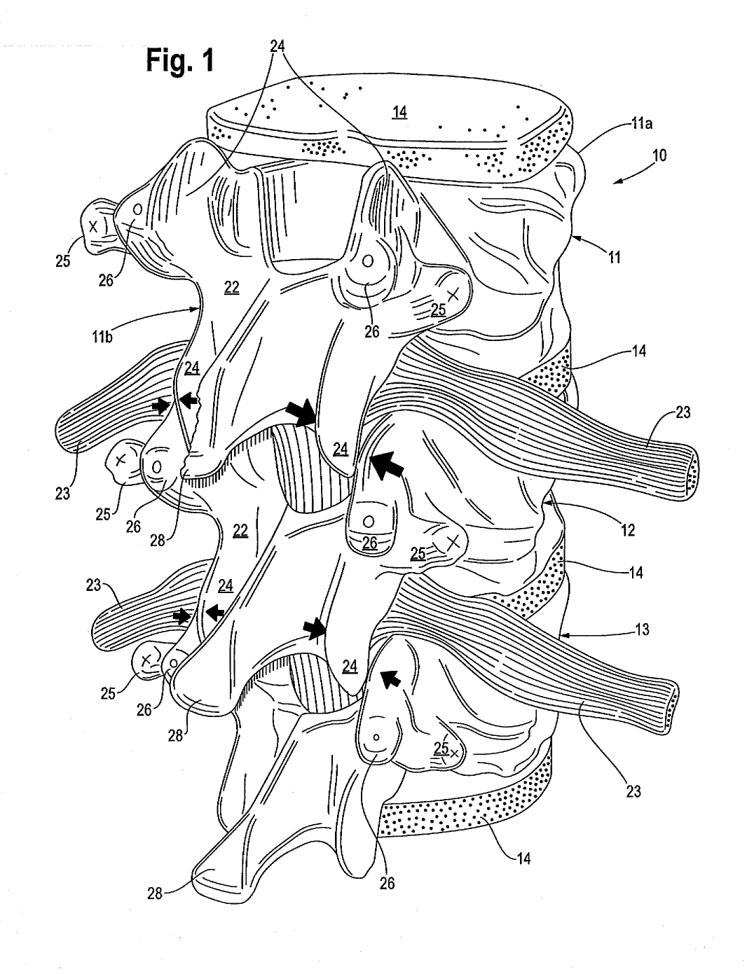

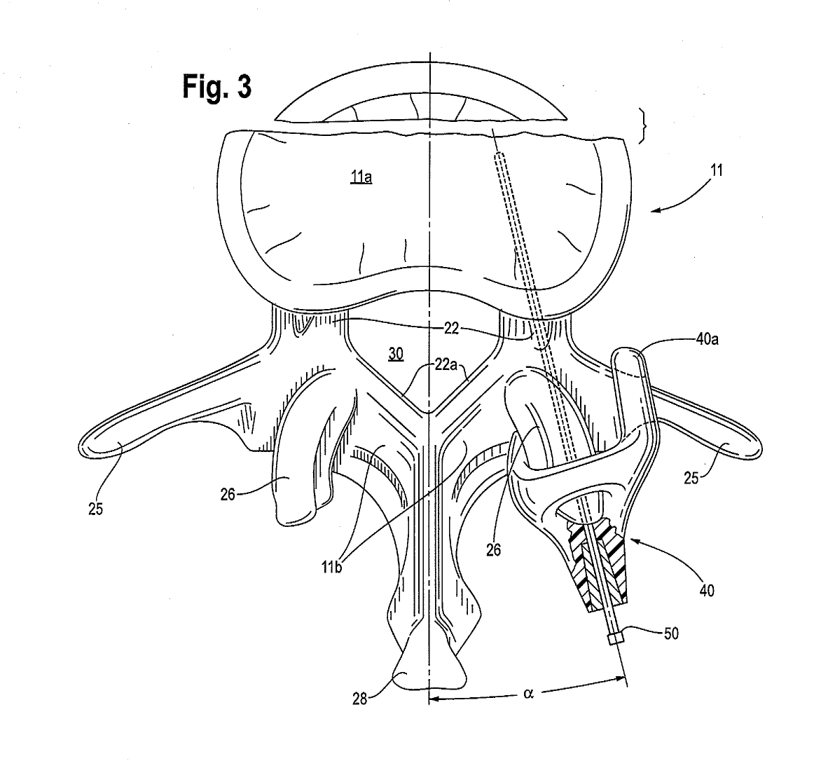

Referring first to FIG. 1, a portion of the human spine in the lumbar region is generally designated by reference numeral 10. Spinal region 10 includes individual vertebrae 11, 12 and 13, each separated by cartilage and muscle loosely represented by reference numeral 14, and spinal nerves 23. (The spinal cord is attached to the spinal nerves and is not shown in FIG. 1. Spinal canal 30 is shown in FIG. 3.) A typical vertebrae 11 consists of an anterior (front) segment 11a, which is the vertebral body, and a posterio (back) portion 11b, which is the vertebral (neural) arch, which enclosed the vertebral foramen. Vertebral arch 11b is formed by a pair of pedicles 22 and a pair of laminae 22a, and supports seven processes: four articular 24, two transverse 25, and one spinous 28. The superior and inferior articular facets 26 form the facet joint. As can be seen in FIG. 3, pedicles 22 form the passageway or isthmus between vertebral body 11a, on the one hand, and the posterior elements, facets 26 and transverse processes 25, on the other.

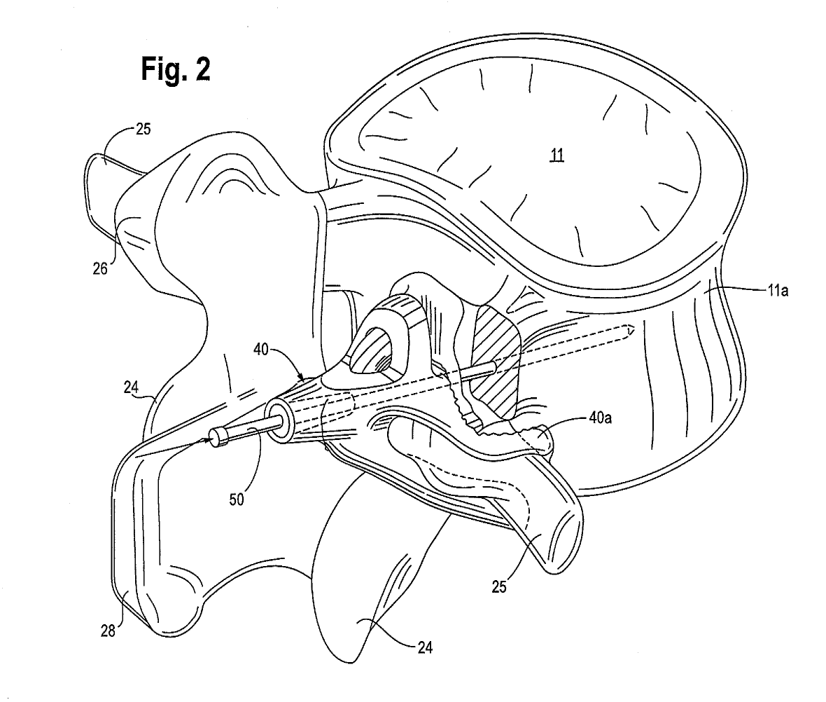

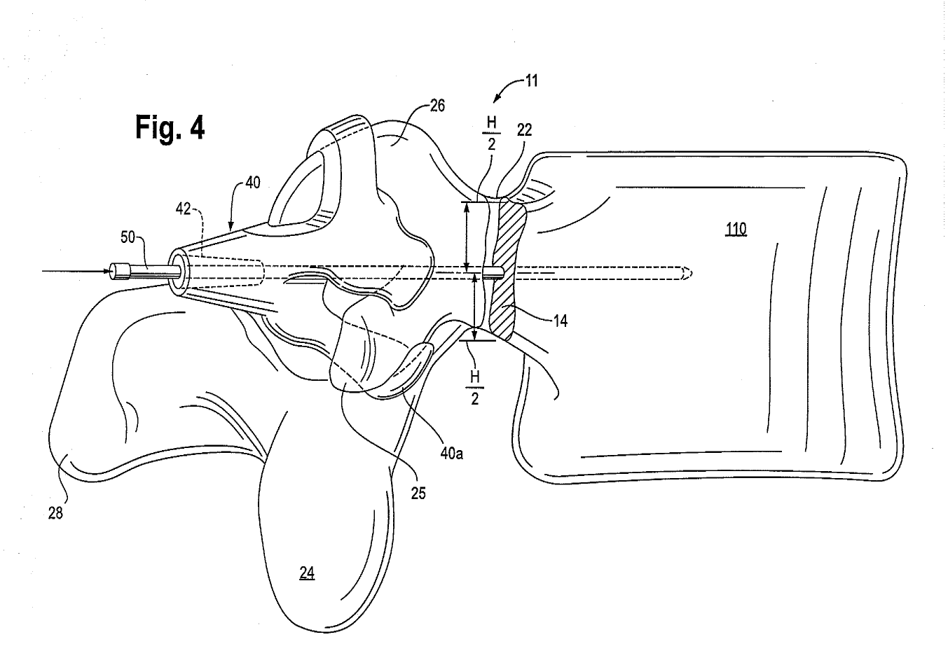

Referring now to FIGS. 2-4, one preferred embodiment of a pedicle implant guide 40 for use in placing a spinal pedicle implant 50 (e.g., the guide wire shown, or a bur, a feeler gauge, a pedicle screw, or other spinal implant) is shown. Pedicle implant guide 40 is preferably made to fit a patient's specific spinal anatomy, and may be manufactured using a rapid manufacturing process such as employed by Materialize of Denmark, using appropriate 3D diagnostic imaging tools such as CT, X-Rays or MRI to make 3D scans of the spinal region in question, such that the guide will precisely fit the contours of the individual patient's vertebrae. Pedicle screw guide 40 preferably includes sleeve 42 which fits within sleeve guide 44 (see FIG. 3). Sleeve 42 and sleeve guide 44 may but need not be restricted to being generally cone-shaped, as shown. Preferably, the outer diameter (OD) of sleeve 42 is constant, while the inner diameter (ID) of sleeve 42 may vary, to permit the use of varying OD implants such as guide wires and pedicle screws, depending on the patient's specific anatomy.

In practice, following 3D image scanning of the vertebral areas in question, a patient-specific, discardable or reusable (depending on preference) pedicle implant guide 40 may be manufactured using a suitable rapid prototyping process and delivered to the surgeon for fitting prior to surgery. The surgeon may fit the guide to a model of the patient's relevant vertebral anatomy. (During the pre-op fitting, the surgeon or assistant may, for example, choose to draw a shape around the guide while fitted to the patient's model; later, during surgery, the surgeon or assistant may again circumscribe the guide (with a marker, for example), this time on the patient's actual vertebrae, and the surgeon may compare the shapes drawn pre-op and during surgery; if the shapes correspond, this will provide another level of confidence for the surgeon that the placement is optimal.)

Assuming the surgeon finds the guide fit acceptable, surgery may then be scheduled, and the guide may also be used during surgery to place first a guide wire and/or bur, and then ultimately a spinal implant such as a pedicle screw. A feeler gauge may be inserted both prior to and following insertion of the guide wire to ensure proper placement of the guide wire, and to confirm that the surgeon has obtained 5-wall bony continuity along the intended implant trajectory. The guide wire may be inserted through the facet and into the pedicle either manually, or using a drill, for example. Importantly, to obtain the appropriate trajectory for the guide wire, a 3-point fixation technique is preferably employed. In one preferred 3-point fixation technique, guide 40 is located into proper position using the medial 26a and lateral 26b boundaries of facet 26, as well as an adjacent transverse process 25, as the 3 points of fixation for the guide. In different areas of the spine, artisans will understand that other appropriate anatomical landmarks may be used to apply 3-point fixation techniques in order to properly orient the guide to provide optimal placement of the spinal implant. 3-point fixation technique in this context has been found to substantially demarcate the alignment, trajectory and angulation of the spinal implant such as a guide wire. After the guide wire is placed and proper placement is indicated, the guide wire may be withdrawn and a fastener such as pedicle screw 50 may be inserted into the hole vacated by the guide wire, such that the screw is optimally located within the pedicle contiguous with the facet.

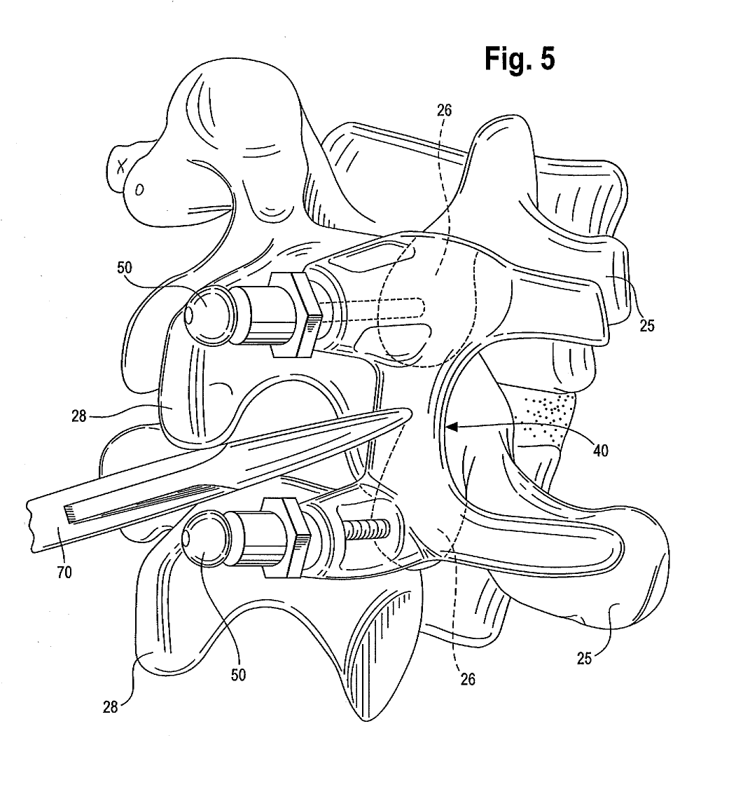

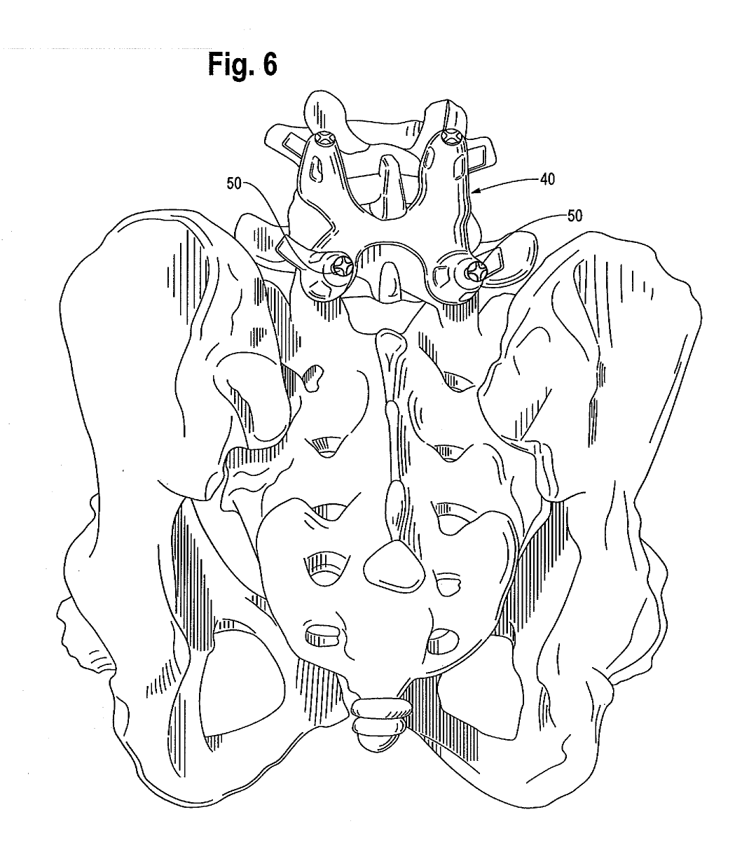

Depending on the physician's preference given the patient's anatomy, multiple pedicle screws may also be secured to a pedicle guide which may be configured in various ways to facilitate placement, such as the dual and quad/H-shaped guides shown in FIGS. 5 and 6, respectively. (Forceps 70 or other devices well known to surgeons may be used to manipulate guide 40 into proper location. Once the guide is properly fitted into proper position and orientation, it may be retained there using any suitable custom or adjustable fixation techniques as are well known.

Those of ordinary skill in the art will recognize that the present invention is intended to describe and protect the concept of utilizing three separate anatomical landmarks or reference points to define the optimal trajectory for placement of spinal implant devices, using an anatomically-correct, patient-specific, pre-operative and/or intra-operative guide(s) prepared using an appropriate 3D diagnostic imaging tool, and regardless of individual patient anatomical variations. (It may be desirable to use a pre-op implant guide, and then a modified implant guide for use in the surgical procedure, depending on what was learned during the preoperative planning.) Once the 3 points of fixation have been obtained, the invention will naturally accommodate variations in individual spinal anatomy.

The above description is not intended to limit the meaning of the words used in the following claims that define the invention. Persons of ordinary skill in the art will understand that a variety of other designs still falling within the scope of the following claims may be envisioned and used. For example, while preferred embodiments have involved the lumbar region of the spine, other embodiments still falling within the principles of the present invention and within the scope of the following claims may involve methods of implant placement in other regions of the spine, such as the upper-most and lower-most spinal regions, and using anatomical landmarks other than those referenced above in order to achieve 3-point fixation as discussed here. It is contemplated that future modifications in structure, function, or result will exist that are not substantial changes and that all such insubstantial changes in what is claimed are intended to be covered by the claims.

I claim:

1. A process for utilizing a spinal implant guide made using a diagnostic imaging tool to facilitate implantation of a device in the spine, comprising the steps of:
providing the spinal implant guide having a body designed to conform to certain predetermined, patient-specific anatomical vertebral landmarks; and
fitting the guide to the anatomical vertebral landmarks during a surgical procedure using 3-point fixation technique to thereby substantially demarcate an optimal alignment, trajectory and angulation for the spinal implant device

2. The process of claim 1, wherein the spinal implant device is fitted to the following anatomical vertebral landmarks in order to accomplish its 3-point fixation: medial and lateral boundaries of a spinal facet joint, and an adjacent transverse process of the spine.

3. The process of claim 1, wherein the spinal implant device comprises one or more of the following: a guide wire; a bur; a feeler gauge; or a pedicle screw.

4. The process of claim 1, wherein the guide is sized and shaped to fit a particular patient's vertebral anatomy e based upon biofeedback information provided by a diagnostic imaging tool.

5. The process of claim 4, wherein the diagnostic imaging tool provides a three-dimensional reconstruction of anatomical images of selected portions of the spine.

6. The process of claim 4, wherein the diagnostic imaging tool comprises one or more of the following image scanning devices: MRI; X-Ray; or CT.

7. The process of claim 1, wherein the guide includes a sleeve guide and a sleeve shaped to at least partially fit within the sleeve guide, to facilitate entry and placement of the spinal implant device.

8. The process of claim 7, wherein, among different sleeves provided and used for different patients, an outer diameter of the sleeve is constant, and an inner diameter of the sleeve varies.

9. The process of claim 1, further comprising the step of pre-operatively fitting the spinal implant guide to a patient-specific vertebral model to analyze the fit of the guide.

10. The process of claim 1, wherein the spinal implant guide is disposable after surgical use with a single patient.

11. The process of claim 1, wherein the spinal implant guide is reusable for multiple patients.

12. The process of claim 9, further comprising the step of modifying the spinal implant guide to be used in the surgical procedure based upon feedback provided during the pre-operative use of the guide.

13. A process for making and using a spinal implant guide, comprising the steps of:
preparing the spinal implant guide having a body designed to conform to certain predetermined, patient-specific anatomical vertebral landmarks based upon biofeedback information provided by a diagnostic imaging tool; and
fitting the guide to medial and lateral boundaries of a facet and to an adjacent transverse process, thereby providing the guide with three-point fixation to substantially demarcate an optimal alignment, trajectory and angulation for a spinal implant to be placed into a pedicle contiguous with the facet;

14. The process of claim 13, further comprising the steps of using the guide to optimally locate a guide wire inserted through the facet and into the pedicle, and withdrawing the guide wire and inserting a pedicle screw within a passageway vacated by guide wire.
